(19) United States
(12) Patent Application Publication
(10) Pub. No.: US 2007/0116784 A1
(43) Pub. Date: May 24, 2007

(54) Imatinib combinations for head and neck cancers

(76) Inventors: Timothy Ward, Manchester (GB); Iain Bruce, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/557,330

(22) PCT Filed: Mar. 17, 2004
(86) PCT No.: PCT/EP04/02769

§ 371 Date: Dec. 12, 2006

(30) Foreign Application Priority Data
May 23, 2003 (GB) 0311971.6

(51) Int. Cl. A61K 38/14; A61K 31/506; A61K 31/7072; A61K 31/704; A61K 31/337; A61K 31/66; A61K 31/525

(52) U.S. Cl. 424/649; 514/8; 514/34; 514/109; 514/252.18; 514/49; 514/410; 514/449; 514/251; 514/492; 514/575; 514/283

(57) ABSTRACT

4-(4-methylpiperazin-1-ylmethyl)-N-[4-methyl-3-(4-pyridin-3-yl)pyrimidin-2-ylamino)phenyl]-benzamide of the formula I 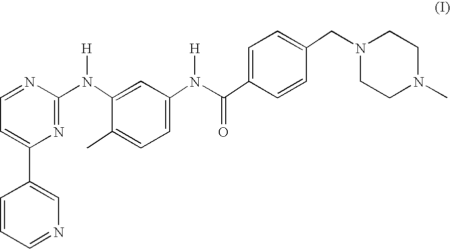 or a pharmaceutically acceptable salt thereof can be used in the treatment of head and neck cancers.

The invention relates to the use of 4-(4-methylpiperazin-1-ylmethyl)-N-[4-methyl-3-(4-pyridin-3-yl)pyrimidin-2-ylamino)phenyl]-benzamide, hereinafter referred as “Compound I”, or a pharmaceutically acceptable salt thereof for the manufacture of pharmaceutical compositions for use in the treatment of head and neck cancers, to the use of Compound I or a pharmaceutically acceptable salt thereof in the treatment of head and neck cancers, and to a method of treating warm-blooded animals including humans suffering from head and neck cancers by administering to a said animal in need of such treatment an effective dose of Compound I or a pharmaceutically acceptable salt thereof.

Treatment of Head and Neck Cancers

The annual number of new cases of head and neck cancers in the United States is approximately 40,000, accounting for about 5% of adult malignancies.

There are three main treatment options for head and neck cancer: surgery, radiation and chemotherapy. One of these therapies, or a combination of them, may be used to treat the cancer. Chemotherapy drugs with single-agent activity in this setting include methotrexate, 5-fluorouracil, herein after abbreviated as 5 FU, cisplatin, paclitaxel, and docetaxel. Combinations of cisplatin and 5 FU, carboplatin and 5 FU, and cisplatin and paclitaxel are also used.

Surgery, radiation and chemotherapy to the head and neck can cause many side effects well known in the art. Improvements in head and neck cancer therapy would include better anti-tumor efficacy, fewer side effects, and lower cost and hospitalization rates, thereby being more amenable to patients.

The complete response of head and neck cancer to systemic therapies is often disappointing.

The instant invention is a response to the need for an alternative therapy in the treatment of head and neck cancers.

It has now surprisingly been demonstrated that head and neck cancers can be successfully treated with Compound I, or pharmaceutically acceptable salt thereof and with Compound I or a pharmaceutically acceptable salt thereof and at least one compound selected from the compounds indicated for the treatment of head and neck cancers.

### LEGEND TO FIGURES

FIG. 1: 3-D synergy plot of the response of Cal27 cells to various combinations of Compound I and cisplatin. Area profiles shown are at the 95% confidence interval.

FIG. 2: 3-D synergy plot of the response of Cal27 cells to various combinations of Compound I and Gemcitabine. Area profiles shown are at the 95% confidence interval.

Epithelial carcinomas of the head and neck arise from the mucosal surfaces in the head and neck area and typically are squamous cell in origin. This category includes tumors of the paranasal sinuses, the oral cavity, and the nasopharynx, oropharynx, hypopharynx, and larynx. Tumors of the salivary glands differ from the more common carcinomas of the head and neck in etiology, histopathology, clinical presentation, and therapy.

Squamous cell head and neck carcinomas can be divided into well-differentiated, moderately well-differentiated, and poorly differentiated categories. Patients with poorly differentiated tumors have a worse prognosis than those with well-differentiated tumors.

The mucosal surface of the entire pharynx is exposed to alcohol and tobacco-related carcinogens and is at risk for the development of a premalignant or malignant lesion, such as erythroplakia or leukoplakia (hyperplasia, dysplasia), that can progress to invasive carcinoma. Alternatively, multiple synchronous or metachronous cancers can develop. In fact, patients with early-stage head and neck cancer are at greater risk of dying of a second malignancy than of dying from a recurrence of the primary disease. Second head and neck malignancies are not therapy-induced but, instead, reflect the exposure of the upper aerodigestive mucosa to the same carcinogens that caused the first cancer. These second primaries develop in the head and neck area, the lung, or the esophagus.

There are six main types of head and neck cancer, named by the part of the body where they begin.

1—Carcinoma of the Lip

Carcinoma of the lip usually presents early and consequently carries a good prognosis. It arises from vermilion border. The lower lip is most commonly affected, i.e. 95%. It predominatly squamous cell carcinomas, with adenoid cystic carcinoma on the inner surface of the lip.

2—Salivary Gland Cancers

Cancer of the salivary gland is a malignant tumor of the tissues of the salivary glands. Most cancerous tumors begin in the largest of the salivary glands, the parotid gland, found on either side of the face in front of the ears and the submandibular glands under the jawbone. Salivary gland tumors can arise from the major, i.e parotid, submandibular, sublingual, or minor salivary glands, i.e. located in the submucosa of the upper aerodigestive tract. Most parotid tumors are benign, but half of submandibular and sublingual gland tumors and most minor salivary gland tumors are malignant. Malignant tumors include mucoepidermoid and adenoid cystic carcinomas and adenocarcinomas.

3—Oral Cavity and Oropharyngeal Cancer

One of the most common types of head and neck cancer is cancer of the oral cavity, i.e. mouth and tongue, and the oropharynx, i.e the middle of the throat. More than 90 percent of oral and oropharyngeal cancers are called squamous cell carcinoma because they begin in the flat, squamous cells in the layer that lines the mouth and throat.

4—Nasopharyngeal Cancer

Nasopharyngeal cancer is a disease that attacks the nasopharynx, the air passageway at the upper part of the throat, i.e. pharynx, behind the nose. There are several types of benign nasopharyngeal tumors: angiofibromas and hemangiomas involve the vascular or blood-carrying system; and tumors in the lining of the nasopharynx that include the minor salivary glands. Malignant nasopharyngeal tumors are cancerous and can invade and damage healthy tissue and organs in other parts of the body. The two main subtypes of nasopharyngeal carcinoma are keratinizing squamous cell carcinoma and non-keratinizing squamous cell carcinoma. Non-keratinizing carcinomas can be further divided into differentiated and undifferentiated carcinoma. Many times carcinomas, e.g. nasopharyngeal carcinomas, also contain immune cells, especially lymphocytes, e.g. lymphoepithelioma, i.e. undifferentiated carcinoma.

5—Nasal Cavity and Paranasal Sinus Cancers

Nasal cavity and paranasal sinus cancers are malignant tumors that begin in the area around the nose. Cancer of the maxillary sinus is the most common paranasal sinus cancer. The nasal cavity and paranasal sinuses are lined by a layer of mucus-producing tissue with the following cell types: squamous epithelial cells, minor salivary gland cells, nerve cells, infection fighting cells, and blood vessel cells.

6—Laryngeal and Hypopharyngeal Cancer

Cancer can develop in any of the three parts of the larynx, i.e. Glottis, Supraglottis or Subglottis. About 95% of all cancers of the larynx and hypopharynx are called squamous cell carcinoma because they begin in the flat, squamous cells that make up the thin, outer layer of these body parts.

Compound I, 4-(4-methylpiperazin-1-ylmethyl)-N-[4-methyl-3-(4-pyridin-3-yl)pyrimidin-2-ylamino)phenyl]-benzamide has the following formula

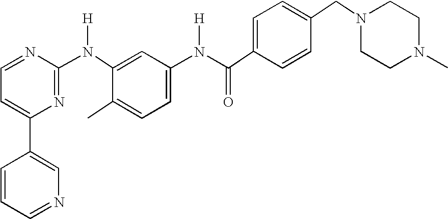

The preparation of Compound I and the use thereof, especially as an anti-tumor agent, are described in Example 21 of European patent application EP-A-0 564 409, which was published on 6 Oct. 1993, and in equivalent applications and patents in numerous other countries, e.g. in U.S. Pat. No. 5,521,184 and in Japanese patent 2706682.

Pharmaceutically acceptable salts of Compound I are pharmaceutically acceptable acid addition salts and they are described in EP-A-0 564 409.

The monomethanesulfonic acid addition salt of Compound I or “imatinib mesylate” and a preferred crystal form thereof, e.g. the beta-crystal form referred herein as “Salt I”, are described in PCT patent application WO99/03854 published on Jan. 28, 1999 hereby incorporated by reference. Possible pharmaceutical preparations, containing an effective amount of Compound I are also described in WO99/03854.

The present invention thus concerns the use of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof, e.g. Salt I, for the manufacture of a medicament for treating head and neck cancers.

The present invention particularly concerns the use of Compound I or a pharmaceutically acceptable salt thereof, e.g. Salt I, for the manufacture of a medicament for head and neck cancers, especially adenoid cystic carcinoma and squamous cell carcinoma.

Pharmaceutical preparation for the treatment of head and neck cancers, especially adenoid cystic carcinoma and squamous cell carcinoma, comprising 4-(4-methylpiperazin-1-ylmethyl)-N-[4-methyl-3-(4-pyridin-3-yl)pyrimidin-2-ylamino)phenyl]-benzamide (Compound I).

The term “treatment” as used herein means curative treatment and prophylactic treatment.

The term “curative” as used herein means efficacy in treating ongoing episodes of head and neck cancers.

The term “prophylactic” means the prevention of the onset or recurrence of head and neck cancers.

The invention relates also to a method which comprises administering a pharmaceutically effective amount of Compound I or a pharmaceutically acceptable salt thereof, e.g. Salt I, to a subject having head and neck cancers. Preferably, Compound I or a pharmaceutically acceptable salt thereof, e.g. Salt I, is administered once daily for a period exceeding 3 months. The invention relates especially to such method wherein a daily dose of 100 to 1000 mg, e.g. 200 to 800 mg, especially 400-600 mg, preferably 400 mg, of Compound I or a pharmaceutically acceptable salt thereof, e.g. Salt I, is administered.

Depending on species, age, individual condition, mode of administration, and the clinical picture in question, effective doses, for example daily doses of about 100-1000 mg of Compound I, preferably 200-600 mg, especially 400 mg, are administered to warm-blooded animals of about 70 kg bodyweight. For adult patients with unresectable head and neck cancers, a starting dose of 400 mg of Compound I daily can be recommended. For patients with an inadequate response after an assessment of response to therapy with 400 mg of Compound I daily, dose escalation can be safely considered and patients may be treated as long as they benefit from treatment and in the absence of limiting toxicities. In another embodiment, the invention also concerns a use or method of treatment as described herein, wherein the administration of the Compound I or a pharmaceutically acceptable salt thereof, e.g. Salt I, occurs before or concurrently with radiation therapy, e.g. external or interstitial radiation and or chemotherapy.

The invention relates to a combination which comprises (a) N-{5-[4-(4-methyl-piperazino-methyl)-benzoylamido]-2-methylphenyl}-4-(3-pyridyl)-2-pyrimidine-amine, designated hereinafter as Compound I or a pharmaceutically acceptable salt thereof and (b) at least one compound selected from the compounds indicated for the treatment of head and neck cancers such as chemotherapy drugs, e.g. melphalan, doxorubicin, cyclophosphamide, leucovorin, epicurin, vincristine, mitomycin C, hydroxyurea, bleomycin, methotrexate, 5 FU, cisplatin, paclitaxel, carboplatin and docetaxel and optionally at least one pharmaceutically acceptable carrier for simultaneous, separate or sequential use, in particular for the treatment of head and neck cancers; a pharmaceutical composition comprising such a combination; the use of such a combination for the preparation of a medicament for the delay of progression or treatment of head and neck cancers; and to a commercial package or product comprising such a combination.

The invention pertains to a method of treating a subject, especially a human, suffering from head and neck cancer, comprising administering to said subject, a combination which comprises (a) Compound I or a pharmaceutically acceptable salt thereof, e.g. Salt I and (b) cisplatin.

The invention pertains to a method of treating a subject, especially a human, suffering from head and neck cancers, comprising administering to said subject, a combination which comprises (a) Compound I or a pharmaceutically acceptable salt thereof, e.g. Salt I, and (b) cisplatin and 5-fluorouracil.

The above-described combinations can be used before surgery or radiation therapy, e.g. external or interstitial radiation, or are given simultaneously rather than sequentially.

Paclitaxel (Taxol®) is a natural product with antitumor activity. TAXOL is obtained via a semi-synthetic process from Taxus baccata. The chemical name for paclitaxel is 5(beta),20-Epoxy-1,2(alpha),4,7(beta), 10(beta), b 13(alpha)-hexahydroxytax-11-en-9-one4,10-diacetate 2-benzoate 13-ester with (2R ,3S )-N-benzoyl-3-phenylisoserine. Paclitaxel may be administered to a human in a dosage range varying from about 50 to 300 mg/m² day. Paclitaxel may be administered to a human in a dosage range varying from about 135 mg/m² to about 175 mg/m² administered intravenously every 3 weeks, or 100 mg/m² every 2 weeks.

Docetaxel may be administered to a human in a dosage range varying from about 25 to 100 mg/m²day. Docetaxel can be administered, e.g., in the form as it is marketed, e.g. under the trademark TAXOTERE™.

Cyclophosphamide may be administered to a human in a dosage range varying from about 50 to 1500 mg/m²day.

Melphalan may be administered to a human in a dosage range varying from about 0.5 to 10 mg/m² day.

Doxorubicin can be administered, e.g., in the form as it is marketed, e.g. under the trademark ADRIBLASTIN™, Doxorubicin may be administered to a human in a dosage range varying from about 10 to 100 mg/m² day, e.g. 25 or 50 mg/m² day.

Cyclophosphamide may be administered to a human in a dosage range varying from about 50 to 1500 mg/m² day.

Vincristine sulfate may be administered parenterally to a human in a dosage range varying from about 0.025 to 0.05 mg/kg body weight*week. Vincristine sulfate can be administered, e.g., in the form as it is marketed, e.g. under the trademark FARMISTIN™.

5-Fluorouracil may be administered to a human in a dosage range varying from about 50 to 1000 mg/m²day, e.g. 500 mg/m² day.

Methotrexate may be administered to a human in a dosage range varying from about 5 to 500 mg/m² day.

Cisplatin may be administered to a human in a dosage range varying from about 25 to 75 mg/M² about every three weeks.

Carboplatin may be administered to a human in a dosage range varying from about 200 to 400 mg/m² about every four weeks. Carboplatin can be administered, e.g., in the form as it is marketed, e.g. under the trademark CARBOPLAT™.

Gemcitabine hydrochloride (not highly recommended as secondary malignancies may occur) may be administered to a human in a dosage range varying from about 1000 mg/week.

It can be shown by established test models that the Compound I or a pharmaceutically acceptable salt thereof, e.g. Salt I, results in a more effective prevention or preferably, treatment of head and neck cancers. Compound I or a pharmaceutically acceptable salt thereof, e.g. Salt I, has significant fewer side effects as a current therapy. Furthermore, Compound I or a pharmaceutically acceptable salt thereof, e.g. Salt I, results in beneficial effects in different aspect of head and neck cancers such as adenoid cystic carcinoma and squamous cell carcinoma.

Compound I or a pharmaceutically acceptable salt thereof, e.g. Salt I, shows an unexpected high potency to prevent or eliminate head and neck cancers because of its unexpected multifunctional activity, and its activity on different aspects of head and neck cancers. The person skilled in the pertinent art is fully enabled to select a relevant test model to prove the hereinbefore and hereinafter indicated therapeutic indications and beneficial effects, i.e. good therapeutic margin, and other advantages mentioned herein. The pharmacological activity is, for example, demonstrated in in vitro and in vivo test procedures, or in a clinical study as essentially described hereinafter. The following Example illustrates the invention described above, but is not, however, intended to limit the scope of the invention in any way.

### EXAMPLE 1

Compound I, e.g. Salt I, in head and neck cancer: a preclinical report Cell Culture: Cells are maintained in M6 medium (50% RPMI and 50% L-15, Sigma) supplemented with 10% heat inactivated fetal calf serum (FCS, Sigma), glutamine and antibiotics. Cell lines are sub-cultured weekly and routinely screened for mycoplasma contamination. All cell lines and primary cultures are found to be negative for mycoplasma. Primary Cell Culture: Tumor material for the short-term culture is obtained routinely as part of the patients planned surgical procedure. The tissue samples are collected and transported in M6 medium. After removal of necrotic material, the tumor is minced to fine slurry using ‘crossed scalpels’. The slurry is then passed through filters of decreasing diameter (100 μm, 40 μm) and added to tissue culture flasks containing M6. The medium is changed regularly and once the tumor cells have proliferated to near confluence the cells are sub-cultured for up to 4 passages. The 3 ACC primary cultures included are referred to as HN1, HN3, HN5.

Cell lines: The human head and neck squamous cell carcinoma cell lines (HNSCC) Cal 27 and FaDu are used, along with the dysplastic head and neck squamous cell line with partially transformed phenotype, Dok. The c-kit positive cell line A549 is added to the panel to act as a control and is treated in the presence and absence of steel factor, a known ligand for c-kit. To investigate the possible role of EGFR status in the response to cisplatin treatment, the EGFR over-expressing human squamous carcinoma cell line, A431 is added to the cell panel.

Growth Inhibition assay: Growth inhibition is measured using a modification of the Sulforhodamine B (SRB) assay for quantifying cellular protein content (Skehan et al., J. Natl. Cancer Inst. (1990), 82:1107-12. Briefly, cells are plated into 96 well plates (Greiner, UK) at 1000 cells/well in 100 μl of medium. The cells are then allowed to adhere for 24 h prior to the addition of the drug. A 2-fold serial dilution of Compound I, e.g. Salt I, is added in triplicate and the plates are incubated for between 5-7 days dependant upon the previously determined growth characteristics of each individual cell culture. The cells are fixed with 100 μl of pre-chilled 10% trichloroacetic acid after removal of the medium. The plates are then chilled at 4° C. for 1 h. Following this, the plates are washed twice with water and stained with 50 μl of 0.4% SRB in 1% acetic acid for 10 min at room temperature. The unbound dye is removed and the plates are washed in 1% acetic acid to further remove unbound dye. The plates are then air dried and the bound dye solubilized in 100 μl of non-buffered 10 mM Tris-base (pH 8.0) and fluorescence determined using a FL500 fluorescence spectrophotometer (Labsystems UK) with wavelength settings Ex 520 nm, Em 590 nm. Growth inhibition curves are plotted and IC₅₀ values determined.

Synergy Assay: Synergy is determined as described by Prichard and Shipman, Antiviral Research, 1990, 14:181-206. Briefly the vertical drug is added in 8 serial two-fold dilutions across the plates and the horizontal drug in 5 serial dilutions plated at 90° to the first drug. This resulted in a matrix combination of 40 doses in duplicate. In this study the vertical drug is always Compound I, e.g. Salt I. The IC₅₀ value for each cell culture as determined for each drug individually is used as a guide to selecting the initial concentrations of each drug combination. After incubation the plates are fixed, stained and the fluorescence read as described previously. A 3-D spreadsheet model is used to process the data generated from two 96 well plates. The data from the experimental drug combination assay are then compared with the predictive additive interaction. Synergy or antagonism is quantified by the volume of the area above (positive volume) or below (negative volume) the additive plane. A positive volume indicating synergy whilst a negative volume indicates antagonism. At the 95% confidence level values up to 25 are considered additive, values between 25-50 indicated minor but significant synergy or antagonism, whilst values between 50-100 are considered moderately synergistic or antagonistic.

Deoxycytidine Kinase Assay: The effect of Compound I, e.g. Salt I, on deoxycytidine kinase (dCK) activity is measured using a modified method of Singhal et al, Oncol. Res. 1992, 4(11-12):517-22. Briefly, cells are pelleted by centrifugation and lysed in bugbuster (Novagen). The cytosolic fraction is removed. To 500 μl of cell lysate is added 75 μl ATP, 8.6 μl of tritiated cytidine ([³H]CTP) and 378 μl of stock buffer (25 μl 3.5 M MgCl₂, 50 μl 0.1 M DTT, 2500 μl 10 mM KCl and 6825 μl double distilled water). The reaction mixture is incubated for 30 min at 37° C. with increasing concentrations of Compound I, e.g. Salt I. The reaction is stopped by boiling for 3 min and the precipitated proteins are sedimented by micro-centrifuge for 60 s. 50 μl aliquots of supernatant are subsequently applied to discs of DEA 81 cellulose paper. Un-phosphorylated deoxycitidine is removed by 3 successive 5 min washes with 1 mM ammonium formate. Finally the discs are washed in water for 5 min followed by dehydration in ethanol for a further 5 min. The discs are then allowed to air dry, placed in scintillation vials and counted in 5 ml of xylene-based scintillant.

Thymidine kinase (TK) may also phosphorylate deoxycytidine and therefore cold thymidine is added to each reaction mixture to a final concentration of 50 mM in order to saturate TK activity.

Receptor Status: The C-Kit receptor status of the primary tumors is established as positive on diagnosis. Further IHC investigations on explanted primary tumor cultures and established cell lines are carried out by the histology department of the Christie hospital.

### Results

Growth Inhibition: The IC₅₀ values for the panel of drugs tested are shown in Table 1. The response of the cell lines and ACC primary cultures to Compound I, e.g. Salt I, is relatively uniform with the IC₅₀ relatively high, (17-33 μM). This micro-molar response is also seen with the two cross-linking agents cisplatin and melphalan (Table 1). In the cell lines Cal27 showed the most sensitivity to this drug. Response to tubulin binding agents is uniform. The ACC primary cultures (HN1, HN3, HN5) appear slightly more resistant to the panel of drugs than the established cell lines. In this group of cells the response to Compound I mirrors that of the established cell lines. The IC₅₀ for Compound I in the c-kit over-expressing cell line A549 is found to be similar to that of the established cell lines and primary cultures at 15 μM. This value is un-altered when these cells are treated in the presence of steel factor.

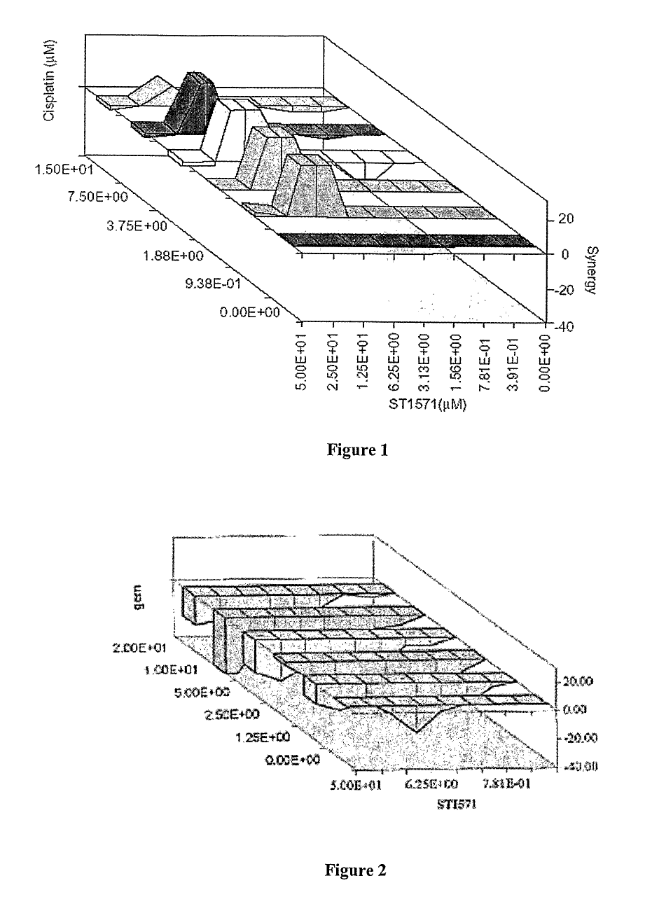

Synergy: The results of the 3-dimensional synergy analysis show a mild but significant degree of synergy with cisplatin in both the DOK and Cal 27 cell lines (Table 2). The Cal27 cell line shows the greatest degree of synergy (FIG. 1) whilst interestingly the FaDu cell line shows no significant synergy with cisplatin despite having a response to both drugs similar to that of the DOK cell line (Table 1). No significant synergistic response is observed in the primary ACC cultures. Likewise no synergistic effects are observed with other combinations of Compound I and other drugs in the panel. Response to cisplatin in epithelial tumors has been associated with EGFR status. Although there is no evidence that Compound I acts on EGFR tyrosine kinase the possibility that the observed synergy may be due to such an interaction is explored using the EGFR over-expressing cell line A431. A mild but significant synergy is observed at a similar level to that of the other established cell lines (synergy=27.6 at 95% confidence interval).

Antagonism: Significant antagonism is observed between gemcitabine and Compound I in all cell lines tested (Table 3, FIG. 2). Since gemcitabine is activated by deoxycytidine kinase (dCK), the effect of Compound I on this enzyme is investigated using cytosol extracts from Cal27 cells. A dose dependant inhibition of dCK activity is observed , with an IC₅₀ of 60 μM (Table 4).

Receptor Status: The morphology of the explanted tumor tissue is observed as epithelial, however the c-kit receptor status is found to be negative. Since the c-kit receptor status of the original tumor is verified at diagnosis as positive, down-regulation of the receptor must have occurred as a response to culture conditions. The established HSCC cell lines are also c-kit receptor negative.

Initial experience with cytotoxic chemotherapy led some clinicians to advocate no role for chemotherapy in ACC. Currently it has become more prevalent to use chemotherapy for symptomatic recurrent and metastatic disease. Single agent response rates are low with the most active agents appearing to be cisplatin, 5-fluorouracil and the anthracyclines. Compound I alone has growth inhibitory effects on a panel of HNSCC cell line despite the lack of a receptor kinase target being expressed in these cells. The original ACC tumor biopsies are established as positive for C-kit receptor expression, however; clearly this expression appears to be down-regulated on adaptation to short term culture. None the less when these primary cultures are exposed to Compound I they show a similar response to that of the HNSCC cell lines. The range over which Compound I inhibits cell proliferation is high, however the c-kit positive cell line A549 show a similar response in both the presence and absence of the c-kit ligand steel factor. It may be that small changes in receptor mediated proliferation in cultured cells are not sufficient to produce significant effects in growth inhibition by drugs targeted to these receptors in vitro. Since Compound I does clearly have an anti-proliferate effect on cells in culture; combinations of existing clinical drugs and Compound I are explored. In the panel of drugs tested most showed no significant interactions except in two of the three HNSCC lines where mild but statistically significant synergy is observed with cisplatin. The EGFR status of many head and neck cell lines, including the ones used in this study, are often positive. The EGFR over-expressing cell line A431 is used as a positive control. Again a mild but statistically significant synergy is observed. The observed antagonism to gemcitabine by Compound I would also appear to be due to an inhibition of deoxycytidine kinase activity. A direct inhibitor of dCK activity would prevent the activation of gemcitabine and thus reduce it effectiveness. Whilst the dose if Compound I required inhibiting dCK activity is high (120 μM) it is less than five fold of the observed IC₅₀ of this drug in these cell lines. Since Compound I binds to the ATP binding site of the abl protein kinase, it is likely that other ATP dependant proteins such as nucleotide transporters may be affected. Gemcitabine is known to be a substrate for nucleotide transporters, however gemcitabine resistance is not related to changes in transport of this drug across the cell membrane. Consequently antagonism of gemcitabine by Compound I is unlikely to be due to effects on these transporter proteins. The data presented here is consistent with the suggestion that antagonism results from dCK inhibition by Compound I.

TABLE 1
IC₅₀ values for both the primary cultures and the cell lines (NT: not tested).

| | Cpd I | Cisp | Adr | Taxol | Mel | 5FU | VBL | Gem | RH1 | MMC | Ca4 |
|---|---|---|---|---|---|---|---|---|---|---|---|
| | μM | μM | μM | ng/ml | μM | μM | ng/ml | nM | nM | μM | nM |
| DOK | 30.05 | 3.7 | 0.02 | 2.7 | 111.4 | 22.0 | 0.3 | 7.06 | 0.33 | 0.06 | 3.32 |
| FaDu | 33.7 | 2.4 | 0.04 | 2.1 | 6.4 | 3.7 | 0.1 | 4.05 | 1.34 | 0.05 | 3.69 |
| Cal27 | 18.1 | 7.4 | 0.02 | 3.8 | 11.3 | 4.4 | 0.2 | 7.00 | 0.11 | 0.05 | 1.46 |
| HN1 | 33.2 | 5.0 | 0.08 | 6.1 | 66.6 | 4.8 | 1.3 | 1.95 | 7.11 | 1.75 | >20 |
| HN3 | 27.1 | 2.8 | 0.06 | 6.1 | 27.5 | NT | 0.9 | NT | 0.86 | 1.16 | NT |
| HN5 | 16.9 | 3.3 | 0.08 | 2.4 | 7.3 | 2.2 | 0.3 | 3.98 | 0.87 | 0.73 | 3.06 |

(NT denotes no results, Cpd I = Compound I, Cisp = cisplatin, Mel = melphalan, 5FU = 5 fluorouracil, VBL = vinblastin, Gem = gemcitabine, RH1 = MMC = mitomycin C, Ca4 = combrestatin)

TABLE 2
Compound I/Cisplatin synergy values for both the
primary cultures and the cell lines.
(Values denote synergy at the 95% confidence interval.)

Cells

| | Dok | FaDu | Cal27 | HN1 | HN3 | HN5 |
|---|---|---|---|---|---|---|
| synergy | 36 | 7 | 63 | 6 | 0 | 6 |

TABLE 3
Compound I/Gemcitabine antagonism values in established cell lines.
Similar values are obtained for the primary cultures (Values denotes
synergy at the 95% confidence interval.)

| | Cells | | | | | |
|---|---|---|---|---|---|---|
| | Dok | FaDu | Cal27 | HN1 | HN2 | HN3 |
| Antagonism | −203 | −334 | −298 | −97 | −86 | −170 |

TABLE 4
The effect of Compound I on deoxycytidine kinase
(dCK) activity in Cal27 extracts

| | Compound I ([μg/ml]) | | | | | | | |
|---|---|---|---|---|---|---|---|---|
| | 0 | 25 | 50 | 75 | 100 | 150 | 200 | 400 |
| % of dCK | 100 | 75.4 | 67.6 | 32.6 | 13.6 | 11 | 9.5 | 2.3 |
| activity | | | | | | | | |
| SEM | 10.8 | 31.8 | 16.6 | 6.4 | 6.2 | 2.7 | 2.8 | 2.3 |

The observed synergy with cisplatin in this study suggests a possible role for Compound I in the management of metastatic and recurrent ACC. In addition a potential adverse combination of Compound I and gemcitabine is reported. Furthermore it would appear that dCK is a hitherto unreported potential target for Compound I.

### EXAMPLE 2

Capsules with 4-[(4-methyl-1-piperazin-1-ylmethyl)-N-[4-methyl-3-[[4-(3-pyridinyl)-2-pyrimidinyl]amino]phenyl]benzamide methanesulfonate, beta-crystal form Capsules containing 119.5 mg of Salt I corresponding to 100 mg of Compound I (free base) as active substance are prepared in the following composition:

| | | |
|---|---|---|
| Active substance | 119.5 | mg |
| Avicel | 200 | mg |
| PVPPXL | 15 | mg |
| Aerosil | 2 | mg |
| Magnesium stearate | 1.5 | mg |
| | 338.0 | mg |

The capsules are prepared by mixing the components and filling the mixture into hard gelatin capsules, size 1.

### EXAMPLE 3

45 patients with head and neck cancer are enrolled in an open label, non-randomized two stage phase 2 trial. Compound I, e.g. Salt I, and cisplatin are administered. Compound I is administered at t daily dose of 800 mg per day (400 mg BID), for a maximum of 12 months. Cisplatin is administered at a dose of 80 mg/m² infusion with pre and post hydration at intervals of 3 weeks to maximum of 6 cycles.

### EXAMPLE 4

Patients with head and neck cancer are enrolled in an open label, non-randomized two stage phase 2 trial. Compound I, e.g. Salt I, and cisplatin are administered. Compound I is administered at t daily dose of 800 mg per day (400 mg BID), for a maximum of 12 months. Cisplatin is administered at a dose of 80 mg/m² infusion with pre and post hydration at intervals of 3 weeks to maximum of 6 cycles. 5-Fluorouracil is administered at a dose of 500 mg/m² day.

Taken together, these results suggest that Compound I or a pharmaceutically acceptable salt thereof, e.g. Salt I, e.g. in combination with at least one compound selected from the compounds indicated for the treatment of head and neck cancers, has an unexpected potential for the treatment of head and neck cancers.

These examples illustrate the invention without in any way limiting its scope.

1. A combination comprising (a) N-{5-[4-(4-methyl-piperazino-methyl)-benzoylamido]-2-methylphenyl}-4-(3-pyridyl)-2-pyrimidine-amine of formula 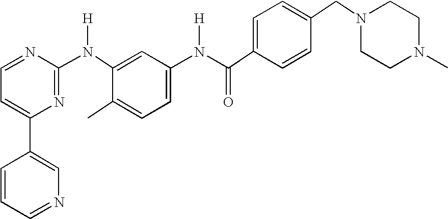
or a pharmaceutically acceptable salt thereof,
and (b) at least one compound selected from melphalan, doxorubicin, cyclophosphamide, leucovorin, epicurin, vincristine, mitomycin C, hydroxylurea, bleomycin, methotrexate, 5 FU, cisplatin, paclitaxel, docetaxel and carboplatin for simultaneous, separate or sequential use in the treatment of head and neck cancer.

2. The combination according to claim 1 wherein N-{5-[4-(4-methyl-piperazino-methyl)-benzoylamido]-2-methylphenyl}-4-(3-pyridyl)-2-pyrimidine-amine is in the form of the monomethanesulfonate salt.

3. The combination according to claim 1 wherein (b) is cisplatin.

4. The combination according to claim 1 wherein (b) are cisplatin and 5 FU.

5. Use of the combination according to claim 1 for the preparation of a medicament for the treatment of head and neck cancer.

6. The use according to claim 5 wherein the head and neck cancers is selected from adenoid cystic carcinoma and squamous cell carcinoma.

7. The use according to claim 6 wherein a daily dose corresponding to 200 to 800 mg of 4-(4-methylpiperazin-1-ylmethyl)-N-[4-methyl-3-(4-pyridin-3-yl)pyrimidin-2-yl-amino)phenyl]-benzamide free base is administered.

8. A pharmaceutical composition comprising the combination according to claim 1.

9. A commercial package comprising the combination according to claim 1 together with instructions for simultaneous, separate or sequential use.

10. A method of treating a subject suffering from head and neck cancer which comprises administering to said subject in need of such treatment a dose, effective against head and neck cancer, of a combination according to 4 claim 1.
